(19) United States
(12) Patent Application Publication
(10) Pub. No.: US 2003/0180365 A1
(43) Pub. Date: Sep. 25, 2003

(54) Preparation containing an oxygen carrier for regeneration of the skin in the case of oxygen deficiency

(76) Inventor: Wolfgang Barnikol, Mainz (DE)

Correspondence Address: KURT BRISCOE, NORRIS, MCLAUGHLIN & MARCUS, P.A., 220 EAST 42ND STREET, 30TH FLOOR, NEW YORK, NY, 10017, US

(21) Appl. No.: 10/312,509

(22) PCT Filed: Jun. 2, 2001
(86) PCT No.: PCT/EP01/06328

(30) Foreign Application Priority Data
Jun. 29, 2000 (DE) 100 31 741.3

(51) Int. Cl. A61K 9/14; A61K 38/42

(52) U.S. Cl. 424/487; 514/6

(57) ABSTRACT

The invention relates to an oxygen carrier-containing preparation, which can be applied externally, hemoglobin and optionally myoglobin being incorporated in a preparation in a gel-like consistency. The preparation is suitable for being rubbed into the skin in order to intensify the diffusive supply of oxygen to the skin from the outside in order to regenerate the skin and eliminate the oxygen deficiency. The agent is also suitable for preventing such conditions and is particularly suitable in the case of degenerative, radiation-induced, thermally induced and age-related skin changes, even after the skin has suffered burns, optionally in co-therapy with intravasal oxygen therapy.

The invention furthermore relates to a method for preparing such a preparation and to its use.

The invention relates to an oxygen carrier preparation, which can be administered externally, hemoglobin and optionally myoglobin being incorporated molecularly dispersed in a preparation of gel-like consistency. The preparation is suitable for being rubbed into the skin, in order to intensify the diffusive supply of oxygen to the skin from the outside in order to intensify the regeneration of the skin and eliminate the oxygen deficiency. The agent is also suitable for preventing such conditions and is particularly suitable in the case of degenerative, radiation-induced, thermal and age-related skin changes, even after the skin has suffered burns, optionally in co-therapy with intravasal oxygen therapy.

The invention furthermore relates to a method for producing such a preparation and to the use of such a preparation.

A series of degenerative changes in the skin are caused by chronic oxygen deficiency. One such deficiency is caused when the blood no longer flows adequately through the skin. This happens either due to a constriction of the small arteries—the blood-supplying vessels—or due to an obstruction of veins—the veins are the discharging vessels of the organism; especially the legs are affected.

In this connection, the clinician is aware of particular syndromes, for example, the chronic, peripheral, occlusive disease with its four different stages according to Fontaine or the diabetic angiopathy, the cause of which is arteriosclerosis, or also a chronic venous insufficiency, that is, a malfunction of vessels.

Chronic oxygen deficiency finally leads to tissue destruction of the skin, also in the form of gangrene or of Ulcus cruris, so-called open sores of the leg. If the oxygen supply is marginal, as it frequently is in older persons, relatively brief compression anemias, which occur when a patient is confined to bed, or only slight bumps lead to the rapid decomposition first of the skin and then also of the underlying tissue, which is referred to as decubitus. It would be of advantage if preventative methods could be employed here to prevent pathological and painful conditions. A detailed explanation of the dermatological clinical practice is found in Braun-Falco, “Dermatologie und Venerologie”, Springer-Verlag, ISBN 3-54053542-X.

A further, important problem area is skin damage after irradiation. In this case, inflammatory and degenerative changes are found. It can be assumed here also that changes can be restrained by an improved, diffusive supply of oxygen to the skin from the outside and a prophylactic therapy is also possible here.

A third important problem area is the damage suffered by skin after bums. Here also, an intensified, diffusive supply of oxygen from the outside can help the skin to regenerate better and faster.

The visible, outer layer of skin consists of about 15 layers of keratinized, that is dying, very flat cells (horny cells). These layers (Stratum corneum) in the normal skin are about 12 μm thick. This corresponds approximately to the diameter of round body cells. The horny cells peel off continuously and are formed by the division of the Stratum germinativum below. Above this, is the Stratum granulosum. These two vital cell layers of the epidermis together are about 25 μm thick. With that, as far as geometric relationships are concerned, intra tissue relationships exist for the oxygen diffusion, since the supply region of a capillary, that is, the smallest type of vessel of an organism, has a depth of approximately 50 μm. It takes about one month for the basal cell of the Stratum germinativum at the surface to be rejected as a keratinocyte from the skin surface.

Under the epidermis, there is the dermis, which arches in the form of many papillae into the epidermis and, in each papilla, there is a supplying capillary with its arterial and venous ends. From this capillary, the oxygen diffuses outward to the lower vital layer of the epidermis, the above-mentioned Stratum germinativum. However, the epidermis receives the necessary oxygen not only from the inside, but also, as was shown, for example by Groβmann et al., Adv. Physiol. Sci. 25 (1981): Oxygen Transport to Tissue, 319-320 or L. R. Fitzgerald, Physiol. Rev. 37(1957): 325-336), to the extent of about 50% diffusively from the outside.

If now, as stated above, the supply of oxygen is deficient due to pathological vascular changes, there is the problem and the objective of ensuring that this deficiency is compensated for otherwise.

It is therefore an objective of the present invention to develop an agent, which increases the external supply of oxygen to the skin and with which the diseases named above can be treated and prevented.

Pursuant to the invention, this objective is accomplished by a preparation, wherein especially a solution of an oxygen carrier, especially hemoglobin or hemoglobin and myoglobin are incorporated molecularly dispersed in a solution containing water and optionally salts into a preparation of gel-like consistency, a gel-forming substance being present in a concentration of 0.1 to 20% and preferably of 0.1 to 8%. In particular, the total solution is incorporated in a gel, such as an inorganic gel (bentonite, silica gel) or an organic gel, such as a gel based on polyacrylic acid, gum arabic, pectin alginates, methylcellulose, hydroxyethylcelulose, starch and carboxymethylcellulose. Preferably, the gel is a hydrogel selected from anionic polyacrylates, especially Carbopol® of the various types, such as Carbopol 940 and 940P. The gel does not contain any fat.

As salts, natural electrolyte components can be present in the solution of the hemoglobin/myoglobin, such as sodium chloride, potassium chloride and sodium bicarbonate, especially in the physiological amounts of 125 mM for sodium chloride, 4.5 mM for potassium chloride and 2.0 for sodium bicarbonate.

The gel used also contains preservatives, such as dibromohexamidine, dihydroacetate acid, 4-hydroxy benzoic acid, benzoic acid, propionic acid, salicylic acid, sorbic acid, formaldehyde, paraformaldehyde, o-phenylphenol, inorganic sulfites and bisulfites, sodium iodate, chlorobutanol and formic acid; in the case of acids, their esters and salts also come into consideration. The preservatives may be present in amounts of 0.15-0.25% or as described below.

Preferably, preservatives are selected from methyl 4-hydroxybenzoate and propyl 4-hydroxy-benzoate, for example, in amounts of 0.15-0.25%, especially of 0.05 to 0.2% and particularly of 0.09-0.17%. Furthermore, humectants, such as sodium lactate, glycerol, propylene glycol, sorbitol and PCA (pyrrolidonecarboxylic acid) may preferably be contained in an amount of 5-15%. Especially preferred are methyl 4-hydroxy-benzoate and propyl 4-hydroxy-benzoate as preservatives and glycerol, propylene glycol and sorbitol as humectants, especially in each case mixtures hereof, such as 1:1 in the case of preservatives and 1:1:1 in the case of humectants. A specially preferred, inventive preparation of gel-like consistency comprises hemoglobin or hemoglobin/myoglobin (approximately 0.1 to 30% and especially 0.1 to 20%, based on the total weight), 5-15% by weight of humectant, 0.15-0.25% of preservative and 0.1 to 20% and especially 0.1 to 8% of Carbopol. Especially preferred are preparations based on 1 to 5% of Carbopol®, 0.02 to 0.08% by weight of propyl 4-hydroxy-benzoate and 0.07 to 0.15% by weight of methyl 4-hydroxy-benzoate and 8 to 12% of glycerol, propylene glycol and/or sorbitol (1:1 or 1:1:1). Preferably, hemoglobin or hemoglobin and myoglobin are present in amounts of 2-8%. In each case, the amounts given are in percent by weight.

Preferably, the hemoglobin in the inventive preparation is a pig hemoglobin (0.1 to 20% and especially 2.8%), which has been stabilized with carbon monoxide (CO). The preparation of such a stabilized hemoglobin is described in the German patent 1 970 103.7, which corresponds to the U.S. Pat. No. 5,985,332. According to the latter, hemoglobin/myoglobin can be converted completely, by equilibration with carbon monoxide, to carboxy hemoglobin/myoglobin, which is stable and does not have to be deligandized before a further intravalen use. Modified hemoglobin can also be carbonylated.

The oxygen carrier is activated by exposing the skin, on which the gel has been applied, to oxygen.

As already mentioned, the hemoglobin can be present in a mixture with myoglobin, the latter being present especially in amounts of 0.1 to 50% by weight, based on the amount of hemoglobin. Preferably, myoglobin and hemoglobin are used in amounts of 50 to 70% hemoglobin and 50 to 30% myoglobin and especially of 75 to 90% hemoglobin and 25 to 10% myoglobin. The percentages are given here as percentages by weight.

As stated, the hemoglobin or hemoglobin and myoglobin are present in certain concentrations in the molecularly dispersed forms, described pursuant to the invention, in a water-binding and deep-acting gel-like preparation, such as a gel.

Surprisingly, pursuant to the invention, with the help of hemoglobins or hemoglobin and myoglobin, incorporated in this manner in gel or gel-like solution structures, especially of modified and unmodified (native), molecularly dispersed hemoglobins or hemoglobin and myoglobin, a facilitated diffusion and, with that, an increased transport of oxygen from the outside through the epidermis is possible. When native hemoglobins are used, their sigmoidal binding characteristics are advantageous since, as a result of these, oxygen from the air can be bound and thus stored and, at the same time, emitted effectively and diffusively, according to Fick's Law, to the vital cell layers of the epidermis. Myoglobin has only a quarter the molecular weight of hemoglobin, so that, if it is used in addition, the advantage arises that an even deeper penetration of the oxygen-transporting molecule into the skin is possible.

The binding of oxygen by hemoglobins can be characterized adequately by two quantitative parameters. These are the so-called oxygen partial pressure at half saturation (p50) of the hemoglobin in question with oxygen, which is an inverse measure of the average affinity of the oxygen for hemoglobin, and the so-called HILL index (n50), which represents a measure of the sigmoidal character of the oxygen-binding curve of the hemoglobin. Under physiological conditions, this index of human hemoglobin in blood is about 2.6 and should be kept as large as possible in all preparations. On the other hand, the p50 value should be optimized.

With the help of effectors of oxygen binding, this can be improved even more with the inventive preparation. Preferably therefore, for the preparation of the solution of the oxygen carrier, known natural effectors, such as 2,3-diphosphoglycerate or artificial effectors, such as inositol hexaphosphate or mellitic acid in an equivalent to 3-fold amount and especially in about an equivalent amount, based on the hemoglobin or hemoglobin plus myoglobin are added (Barnikol et al. Funkt. Biol. Med. 2 (1983) 245-249). Natural effectors, which do not react chemically, but are linked conformatively to the hemoglobin/myoglobin, are described, for example in Lehninger et al, “Prinzipien der Biochemie”, Spektrum-Verlag, 1994.

Moreover, hemoglobin or myoglobin, also preferably, also additionally to the above-name effectors, can be modified chemically by effectors, which are linked covalently to the hemoglobin. These include, for example, pyridoxal-5-phosphate. The preparation of such modified hemoglobins is described in Kothe et al., Surgery 161 (1985), 55 583-599. Alternatively, 2-nor2-formyl-pyridoxal-5-phosphate (van der Plas et al., Transfusion 27 (1985) 424-430) may also be used as effector. Further references may be found in Rudolph A. S., et al. (editors), Red Blood Cell Substitutes, Basic Principles and Clinical Applications, Marcel Dekker, New York, et al. 1998, Tsuschida, E. (editor): Blood Substitutes: Present and Future Perspectives, Elsevier Science, Amsterdam 1998, Chang, T. M. S. (author and editor), Blood Substitutes, Principles, Methods, Products and Clinical Trials, Volumes 1 and 2, Karger Landes, Basel, et al. 1997 and 1998; see also EP 0 528 841, where the pyridoxylation of hemoglobin is described. Covalently linking effectors can be used for hemoglobin as well as for myoglobin.

Surprisingly, the properties of the molecularly dispersed hemoglobins are optimized with the help of the inventive preparation, so that an advantageous, external supply of oxygen to the skin becomes possible for the most effective percutaneous diffusion of oxygen. Moreover, due to the inventive preparation, in which hemoglobin/myoglobin are incorporated in a water-binding and deep-acting gel-like structure, especially in a gel, and the water of the gel-like structure swells the upper skin layers, the resistance to the diffusion of oxygen is advantageously reduced.

Such hemoglobin/myoglobin-containing gel like preparations, which transport oxygen with the help of the mechanism of facilitated diffusion, are of great interest not only medicinally, but also from the point of view of cosmetics. After all, the ageing process of the skin, is affected decisively also by the availability of oxygen to the vital, highly active cell layer of the epidermiis.

For this reason, the hemoglobin-containing or hemoglobin/myoglobin-containing preparations are also particularly suitable as agents for the treatment of age-related oxygen-deficiency conditions of the skin, aside from the treatment of oxygen deficiency conditions in general or of skin changes caused by permanent generative, and/or radiation and thermal factors, preventatively as well as therapeutically, especially also as simultaneous co-therapy with intravasal use of artificial oxygen carriers.

In particular, human hemoglobin, pig hemoglobin or bovine hemoglobin can be used as hemoglobin. The nature of the myoglobin is also variable; it can be obtained from different animal species, such as the dog, the sheep, the horse or the whale. The hemoglobins can be used as native hemoglobins, or preferably, as described, provided with an effector, as described above, and/or protected against oxidation. Moreover, the preparation may have preferably a natural, conformatively linked effector, as described above. As protection against oxidation, hemoglobin as well as myoglobin may, for example, be carbonylated, that is, provided with carbon monoxide (CO) and accordingly stabilized.

If the inventive preparation is brought into the skin in a stabilized form, the hemoglobin or the hemoglobin and myoglobin are reactivated as oxygen binder by a brief, approximately half-hour exposure to pure oxygen, that is, the stabilizer is removed. From then on, the dissolved, artificial oxygen carrier, diffusively reinforced, transports oxygen also from the air, which contains only 20% by volume of oxygen. Preferably, therefore, the hemoglobin or myoglobin used is protected against oxidation, that is, is stabilized.

Alternatively, the oxygen-transporting hemoglobin-myoglobin can also be used without stabilization (protection against oxidation). Although such a preparation cannot be kept for the same length of time as one stabilized with CO, it has the advantage that it can act directly with pure oxygen without a prior activation.

The unstabilized product therefore is more suitable for household application, whereas the stabilized can be used especially for an ambulant or in-patient first therapy.

The above-mentioned hemoglobins/myoglobins are known as such and described, for example, in “Prinzipien der Biochemie” of Lehninger, Nelson, Cox (Spektrum Verlag).

The hemoglobins and myoglobins used can alternatively also be linked covalently particularly to polyalkylene oxides, as described in U.S. Pat. Nos. 4,179,337, 5,478,805 and 5,386,014 and the European patents 0 206 448 and 0 67 029. Such a linkage improves the tissue compatibility of the products.

Covalent linkages of polyalkylene oxides to proteins, especially also to (uncrosslinked) hemoglobin, are known and described in the literature (the state of the art is described comprehensively by J. M. Harris (editor): Poly(Ethylene Glycol) Chemistry: Biotechnical and Biomedical Applications, Plenum, New York et al. 1992). In very many of these methods, the polyalkylene oxide is linked over a molecular bridge (“spacer”), which is formed, for example, by a bifunctional linking agent. Strictly speaking, a linkage product of a polyalkylene oxide with a linkage agent is linked to the protein in these cases.

For the covalent linkage of the polyalkylene oxides (polyalkylene glycols), preferably those derivatives of the polyalkylene oxides are used, which contain a linking agent, which is already bound covalently with a functional group, which enters into a direct chemical reaction with amino, alcohol or sulfhydryl groups of the hemoglobins with the formation of covalent linkages of the polyalkylene oxides, such as polyalkylene oxides with reactive N-hydroxysuccinimidyl ester, epoxide (glycidyl ether), aldehyde, isocyanate, vinylsulfone, iodoacetamide, imidazoyl formate or tresylate groups, etc. Many such monofunctionally activated polyethylene glycols are commercially available. Alternatively, polyalkylene oxides, which have not been activated, can initially be activated chemically in any further, suitable manner or, possibly after an additional, necessary derivatization, be linked to the hemoglobin by chemical linking agents, for example, by a chemical reaction with bromocyan, a carbodiimide such as 1-ethyl-3-(3-dimethylaminopropyl)carbodiimide or N,N′-dicyclohexylcarbodiimide, cyanuric chloride (polyethylene glycol activated with the latter, 4,6-dichloro-s-triazine-polyethylene glycols, are also commercially obtainable), or other known linking agents, such as 2,2′-dichlorobenzidine, p,p′-difluoro-m,m′-dinitrodiphenylsulfone, 2,4-dichloronitrobenzene, etc. (overview in Harris, J. M. (editor): Poly(Ethylene Glycol) Chemistry: Biotechnical and Biomedical Applications, Plenum, New York et al. 1992).

As polyalkylene oxides, especially polyethylene glycols (polyethylene oxides), polypropylene glycols (polypropylene oxides), as well as copolymers of ethylene glycol (ethylene oxide) and propylene glycol (propylene oxide) and especially certain derivatives of the latter are suitable.

As already mentioned, the linking of polyalkylene oxides to proteins is already known, (for example, U.S. Pat. No. 4,179,337 (1979); “Non-immunogenic Polypeptides”, especially also to hemoglobins, namely also to artificial oxygen carriers based on modified hemoglobins (U.S. Pat. No. 5,478,805 (1995): “Fractionation of Polyalkylene Oxide-Conjugated Hemoglobin Solution”, U.S. Pat. No. 5,386,014 (1995): “Chemically Modified Hemoglobin as an Effective, Stable, Non-Immunogenic Red Blood Cell Substitute”, EP-A 0 206 448 (1986): “Hemoglobin Combined with Poly(Alkylene Oxide⁵)”, EP-A 0 067 029 (1982): “Oxygen Carrier”). The content of these publications is therefore incorporated here. According to the literature dealing with the linkage of polyalkylene oxides to artificial oxygen carriers based on modified hemoglobins, such linking reactions are carried out only with hemoglobin, which has not been cross linked.

For example, the EPA 0 067 029 describes the linkage of polyalkylene glycol, such as polyethylene/polypropylene glycol or copolymers of ethylene oxide and propylene oxide or of an ether of said glycols to a C₁ to C₁₆ alcohol, an ester of said glycols with a C₂ to C₁₈ carboxylic acid (preferably butyl monostearate) and an amide of glycol and a C₁ to C₁₈ amine (such as propyl stearylamine). As cross-linking agent, N-hydroxysuccinimide, N-hydroxyphthalamide, p-Nitrophenol and pentachlorophenol, for example, are mentioned. Similarly, reactive derivatives of said polyalkylene glycols can be used.

The molecular weight of the polymers (such as polyethers) may be 300-20,000 and especially 750-10,000). Molar ratios and reaction temperatures depend on the respectively described, known conditions (see Examples), such as a 1 to 40-fold excess of polyalkylene oxide/derivative and a pH of 7 to 10.

As already mentioned, hemoglobin/myoglobin, may also be connected with effectors, such as pyridoxal-5′-phosphate, pyridoxal-5′-sulfate.

The EPA 0 206 448 also describes the linking of polyalkylene oxides, such as those named above, which have an amino function and, accordingly, are connected to hemoglobin over an amide bond. The molecule has the formula —CH₂—O—(CH₂)ₙ—CONH HB (n>1 and especially 1-10). In Examples 1 to 5, the linking with derivatized polyethylene glycol, for example, is described, for example, also when pyridoxal-5′-phosphate-hemoglobin is used.

U.S. Pat. Nos. 5,312,808 and 5,478,805 describe the preparation of hemoglobin-containing solutions with polyalkylene oxide-conjugated hemoglobin with a molecular weight of more than 85,000 Dalton (see especially Examples 1-4, in which the reaction conditions are given).

U.S. Pat. No. 5,234,903 discloses (see examples) a hemoglobin, which is linked with a polyalkylene oxide (such as PEG) and may also be linked, especially, over a carbamide bond (urethane). In Examples I to IV, especially the molar amounts and reaction conditions for the linkage of polyethylene glycol to hemoglobin are given.

According to the German Offenlegungsschrift 30 26 398, (not activated) polyethylene glycol is reacted with the 2-fold to 5-fold molar amount of bromocyan (pH 9-10). The residual bromocyan is removed by gel filtration, dialysis, etc. from the reaction mixture and the product is then reacted in an aqueous solution with the required amount of hemoglobin, such as the 0.1-fold to 0.002-fold amount of hemoglobin (pH 7 to 8). Alternatively, the polyethylene glycol is added to benzene and reacted with the 2-fold to 5-fold molar amount of cyanic chloride. The reaction product, polyethylene glycol-4,5-dichloro-s-triazine, is reacted in a buffer solution with the desired amount of hemoglobin, such as 1 to 0.002 moles.

The methods, explained above, can also be used in the case of the other polymers named, as well as with myoglobin.

Preferably, monomeric hemoglobin/myoglobin, especially if deoxygenated, is cross-linked in aqueous electrolytes, such as sodium bicarbonate, sodium chloride, sodium lactate or mixtures hereof) with an excess of polyalkylene oxide, such as polyethylene/polypropylene glycol (oxide), copolymers or derivatives hereof, especially an activated polyethylene glycol, such as methoxy polyethylene glycol-N-hydroxysuccinimidyl propionate (mPEG-SPA) with the desired molecular weight, as described. The excess of reactant is removed by known methods (lysine). Moreover, preferably an effector can be linked, preferably covalently, as described, and/or especially an effector of the type named above, especially one which also acts only conformatively, may be added to the solution later. A hemoglobin/myoglobin, prepared as described above, may be purified, for example, chromatographically (for example, by preparative, volume exclusion chromatography, for example, on Sephadex G-25), or by centrifugation, filtration or ultrafiltration and processed further subsequently in the manner described to the inventive gel, purification methods also being described in the above-mentioned publications (see also Culring, J. M.: Methods of Plasma Protein Fractionation, Academic Press London, 1980, or EP-A 0 854 151, EP-A 95 107 280). Optionally, the product is stabilized by carbonylation.

Alternatively, native hemoglobin and myoglobin, which have not been modified and, in particular, may be protected against oxidation preferably by carbonylation, are used, the oxygen carrier solution having an effector, which is not chemically reactive, especially 2,3-diphosphoglycerate, as indicated, in an amount ranging from equivalent up to a 3-fold excess, an equivalent amount relative to the hemoglobin/hemoglobin/myoglobin being preferred. Furthermore, with pyridoxal effectors, chemically modified hemoglobin can also be used, as described by Kothe and van der Plas. For this purpose, hemoglobin is reacted with the appropriate effectors named and optionally carbonylated. Preferably, an effector, which is not chemically reactive, can be added to the solution.

Pursuant to the invention, the use of deoxygenated, optionally carbonylated human or, especially, pig hemoglobin, which has not been modified, and of appropriate, deoxygenated dog, sheep or horse myoglobin, which has not been modified, is preferred. The pharmaceutical preparation can be prepared, for example, as follows:

To begin with, a gel-forming substance, such as a hydrogel, preferably with a deep, skin action, preferably of the anionic polyacrylate type (such as Carbopol®), is dissolved in aqua conservans. Aqua conservans can be obtained from the pharmacy or prepared in accordance with the NRF (Neues Rezept Formulatorium), page 6, where the composition of aqua conservans is given. For this purpose, a purified water is used, to which the preservative, especially 0.02 to 0.8 parts and preferably however 0.025 parts by weight of propyl 4-hydroxybenzoate, as well as 0.07 to 0.15 parts by weight and preferably 0.075 parts by weight of methyl 4-hydroxybenzoate is added.

To prepare the paintable gel, 0.1 to 50 g and preferably 1 to 20 g of a gel-forming substance, such as hydrogel, especially 1-5 g of Carbopol® are added to 1 L of aqua conservans and dissolved.

As humectant, for softening the skin, between 5% and 15% by weight of, for example, glycerol, propylene glycol or preferably 8 to 12% of a 70% sorbitol solution according to DAB 9 are added. Alternatively, two or three of the humectants mentioned can be added in the (total) amount given, especially of between 8 and 12% by weight, preferably in equal parts.

The gel is formed after reactants, such as bases, especially sodium hydroxide solution (NaOH) have been added in amounts of 2 to 25 mL of normal sodium hydroxide solution and preferably however in amounts of 6 to 12 mL of 1N NaOH.

Subsequently, a concentrated solution of the hemoglobin or of the hemoglobin and myoglobin, especially of the human, pig or bovine hemoglobin and of bovine, sheep or horse myoglobin, which is not modified or preferably also is chemically modified or provided with an effector, which is not chemically reactive, in a concentration range between 150 and 450 g/L and preferably between 300 and 400 g/L, which preferably has been carbonylated completely by being shaken with pure carbon monoxide (CO), is mixed homogeneously in such a manner into the finished gel composition, that the gel has a hemoglobin/myoglobin content between 0.1 and 20% and preferably between 2 and 8%. As mentioned, up to 50% by weight of myoglobin can be mixed in with the hemoglobins.

In the event that the preparation is prepared for domestic and independent follow-up therapy, the carbonylation of the hemoglobin/myoglobin is omitted.

In contrast to a treatment with pure oxygen at an elevated pressure, the advantage of this type of oxygen therapy, namely, by means of a facilitated diffusion by means of hemoglobin-containing topical applications, is that oxygen can be supplied here in large amounts to the cells in question in large amounts in a “low-pressure” form. Oxygen under a high partial pressure is radically aggressive and has a toxic effect, as has long been known from intensive medicine.

The invention is described in greater detail by means of the following example.

### EXAMPLE

### Preparation of an Oxygen-Transporting Gel, Which Contains Pig Hemoglobin

Methyl 4-hyydroxybenzoate (1.5 g) and 0.5 g of propyl 4-hydroxy benzoate were dissolved in doubly distilled water and made up to 1 L with the latter (:“DAC”).

Carbopol® 940 (5.0 g) is stirred with 45 mL of glycerol and 45 mL of 1,2-propylene glycol. DAC (850 mL) is the added to the mixture, followed by 350 mL of doubly distilled water and 38 mL of 1 M NaOH to form the gel. A 280 g/L pig hemoglobin solution (280 mL) could then be mixed homogeneously into the gel. The hemoglobin was previously liganded to the extent of 99% with carbon monoxide.

1. An externally applicable preparation, containing an oxygen carrier, wherein the oxygen carrier is incorporated, molecularly dispersed, in a preparation of gel-like consistency.

2. The preparation of claim 1, wherein the oxygen carrier is selected from hemoglobin or hemoglobin and myoglobin.

3. The preparation of claims 1 or 2, wherein hemoglobin or hemoglobin and 0.1 to 50% by weight of myoglobin, based on the amount of hemoglobin, are contained.

4. The preparation of one of the claims 1 to 3, wherein the hemoglobin or myoglobin and hemoglobin are incorporated in a gel.

5. The preparation of claim 4, wherein a hydrogel, selected from anionic polyacrylates, is present as gel.

6. The preparation of claim 5, wherein Carbopol® is present as gel.

7. The preparation of one of the claims 1 to 6, wherein the preparation contains additives, selected from preservatives and humectants,

8. The preparation of one of the claims 1 to 7, wherein the preparation contains 5 to 15% of humectant, 0.15 to 0.25% of preservative, a gel-forming substance in an amount of 0.1 to 20% and the hemoglobin or myoglobin and hemoglobin in a concentration of 0.1 to 20%, based on the total amount.

9. The preparation of one of the claims 1 to 8, wherein human, pig or bovine hemoglobin is incorporated as hemoglobin and horse, dog or sheep myoglobin is incorporated as myoglobin.

10. The preparation of one of the claims 1 to 9, wherein native or chemically modified hemoglobin or myoglobin and hemoglobin and/or hemoglobin or myoglobin, protected against oxidation, is/are incorporated as hemoglobin or hemoglobin and myoglobin.

11. The preparation of claim 10, wherein hemoglobin or myoglobin, protected by carbonylation against oxidation, is incorporated.

12. The preparation of claims 10 or 11, wherein the hemoglobin or myoglobin and hemoglobin is linked covalently with a polyalkylene oxide and/or provided covalently and/or conformatively with a natural and/or an artificial effector.

13. The preparation of one of the claims 1 to 12, wherein the hemoglobin or myoglobin is cross-linked with a polyalkylene oxide, especially polyethylene oxide or propylene oxide or copolymers hereof.

14. The preparation of one of the claims 1 to 13, wherein it contains 2 to 8% of deoxygenated pig hemoglobin, 1 to 5% of Carbopol®, 0.02 to 0.08 parts by weight of propyl 4-hydroxybenzoate as well as 0.07 to 0.15 parts by weight of methyl 4-hydroxybenzoate, 8 to 12% by weight glycerol, propylene glycol and/or sorbitol in an aqueous phase.

15. The preparation of one of the claims 1 to 14, wherein an effector, which is not chemically reactive and is selected from 2,3-diphosphoglycerate, inositol hexaphosphate, mellitic acid or mixtures thereof, is incorporated in equivalent amounts, based on the hemoglobin or the hemoglobin and myoglobin.

16. A method for the preparation of an oxygen carrier-containing preparation of one of the claims 1 to 15, wherein a gel-forming substance is incorporated in water, additives optionally are added, subsequently the gel formation is initiated by the addition of reactants and then a hemoglobin solution or a myoglobin solution and hemoglobin solution, having a concentration ranging from 150 to 450 g/L, is added in such a manner, that the gel contains 0.1 to 20% of hemoglobin or hemoglobin and myoglobin.

17. The method of claim 16, wherein a hydrogel is added as gel-forming substance and NaOH as reactant.

18. The method of one of the claims 16 or 17, wherein Carbopol® is added as gel-forming substance.

19. The method of one of the claims 16 to 18, wherein, as additive, preservatives, selected from methyl 4-hydroxybenzoate and propyl 4-hydroxybenzoate or mixtures hereof, and humectants, selected from sorbitol, propylene glycol or glycerol or mixtures hereof, are added.

20. The method of one of the claims 16 to 19, wherein native pig hemoglobin, liganded with carbon monoxide, is added as hemoglobin.

21. The method of one of the claims 16 to 20, wherein hemoglobin or hemoglobin and myoglobin, cross-linked with a polyalkylene oxide selected from polyethylene oxide and polypropylene oxide or copolymers thereof, is added.

22. The use of a preparation of one of the claims 1 to 15 or prepared according to claims 16 to 21 for preparing an agent for the external treatment and/or prevention of oxygen deficiency conditions in the skin.

23. The use of claim 22, wherein the oxygen deficiency is caused by degenerative and/or radiation-induced or thermally induced skin changes.

24. The use of a hemoglobin-containing preparation of one of the claims 1 to 15 or prepared according to one of the claims 16 o 21 for the preparation of an agent for the treatment and/or prevention of age-related conditions of oxygen deficiency in the skin.

25. The use of one of the claims 22 to 24, wherein a stabilized gel preparation is used and, in order to activate the hemoglobin/myoglobin-hemoglobin as oxygen carrier, the skin is gassed with pure oxygen.

26. The use of one of the claims 22 to 24, wherein a hemoglobin or hemoglobin and myoglobin, which has not been stabilized, is used for domestic therapy.
